(19) United States
(12) Patent Application Publication
(10) Pub. No.: US 2009/0312690 A1
(43) Pub. Date: Dec. 17, 2009

(54) SELECTIVE PERIPHERAL NERVE PLEXUS IMPLANTABLE INFUSION DEVICE AND METHOD

(76) Inventor: Philip S. Kim, Bryn Mawr, PA (US)

Correspondence Address: BLANK ROME LLP, ONE LOGAN SQUARE, PHILADELPHIA, PA, 19103, US

(21) Appl. No.: 12/543,700

(22) Filed: Aug. 19, 2009

Related U.S. Application Data
(63) Continuation of application No. 10/616,247, filed on Jul. 9, 2003, now abandoned.
(60) Provisional application No. 60/395,302, filed on Jul. 9, 2002.

(51) Int. Cl. A61M 25/06; A61M 25/00; A61N 1/36

(52) U.S. Cl. 604/21; 604/508; 604/500

(57) ABSTRACT

The present invention relates to a closed system and method for selectively infusing anesthetics into a peripheral nerve or plexus. A method of providing long term pain management is disclosed herein. In the method, a catheter is surgically implanted to create an infusion site at a peripheral neural structure. An implantable pump and reservoir are surgically implanted in subcutaneous tissue. The pump is then operated to deliver a predetermined dosage of medication through the catheter into the infusion site, whereby pain management is provided.

### RELATED APPLICATIONS

This application is a continuation of U.S. application Ser. No. 10/616,247, filed Jul. 9, 2003, entitled Selective Peripheral Nerve Plexus Implantable Infusion Device and Method; which application claims benefit of priority of U.S. Application No. 60/395,302, filed Jul. 9, 2002. The above-identified related applications are incorporated herein by reference.

### FIELD OF THE INVENTION

The present invention relates to a method and system of pain management. More particularly, the present invention relates to a closed implantable system for the administration of analgesic medication to peripheral nerves and plexi, and methods of implanting such a system.

### BACKGROUND OF THE INVENTION

Pain management of both medical and surgical patients has been implemented in the past using numerous devices and methods. Conventional methods of pain management involve oral medication, peripheral nerve stimulation, local injections or intrathecal injections of analgesic medication.

For example, Peripheral Nerve Stimulation (PNS) has been conventionally used for the treatment of mononeuropathy. PNS involves stimulation of mononeuropathy with low voltage stimulation of low threshold afferent nerve fibers. Stimulation of the afferent fibers in selected mononeuropathies has involved surgical implantation of a pulse generator with connecting electrode placement adjacent to the symptomatic nerve. However, a shortcoming of PNS systems is that such systems are conventionally limited for use in the treatment of mononeuropathy.

Another example of a conventional pain management technique is intrathecal anesthesia. Intrathecal anesthesia involves the direct administration of analgesic agents into the epidural space. Such a technique involves the insertion of an epidural needle through the ligamentum flavum to access the Central Nervous System (CNS). Conventional intrathecal anesthesia techniques can involve one time or continual injection of analgesic. An implantable pump and reservoir can be used in conjunction with such a technique to permit home administration of anesthetic to the epidural space. Such conventional pumps and reservoirs involve injection of multiple analgesics such as opioids, lidocaine, tetrocaine, alpha 2 antagonists and baclofen to cause decreased afferent conduction of pain in the CNS. The delivery of anesthetics intrathecally; however, has several shortcomings. For example, such a method of drug delivery carries a risk of hypotension from analgesic effects on sympathetic nerves. In addition, such methods involve the risk of hematoma formation, cord compression, subarachnoid injection, headaches and meningitis.

Yet another example of a conventional pain management technique involves the brachioplexus nerve complex, which receives afferent pain fibers from the upper extremities. Conventional methods used to block such afferent fibers with local anesthetics have been performed in upper extremity surgery for operative and post operative pain management. Several approaches have been utilized for the placement of a brachial plexus catheter which subsequently administers anesthetic to the afferent fibers.

One such conventional local analgesic method involves an axillary approach to achieve a brachial plexus blockade. Such a blockade can be achieved through an axillary, subclavian, interscalene or infraclavicular approach. Each such technique involves the verification of catheter placement by electrical stimulation of the plexus prior to administration of anesthetic. Such blockades are conventionally used to manage operative and post operative pain in the upper extremities. A significant shortcoming of such a technique is that, being an inpatient treatment, it is ill-suited for long-term use. An extension of the above conventional local analgesic method involves the use of a portable continuous pump to administer a brachial plexus block on an outpatient basis. Such a system is open because of the location of the pump outside the body; therefore, it has the serious shortcoming of being prone to infection if used on a long-term basis. Thus, such a system is generally discontinued after seven days due to the risk of infection. In addition, such a system carries a risk of the catheter eventually becoming displaced from the targeted neural structure.

Additionally, interpleural, intercostal and paravertebral local anesthetic injections have been administered to patients for the treatment of rib fractures and surgical pain. The use of such a method, however, is unsuitable for long-term treatment because of the requirement for frequent, repeated injections.

Accordingly, and in light of the shortcomings of the conventional methods and systems discussed above, what is needed is a method of administering analgesics to a targeted nerve structure. More particularly, what is needed is a method resistant to infection and therefore suitable for long-term use. Even more particularly, what is needed is a closed system of a drug reservoir, catheter and pump for use in infusing anesthetics to a peripheral nerve or plexus.

### SUMMARY OF THE INVENTION

The present invention relates to a closed system and method for selectively infusing anesthetics into a peripheral nerve or plexus. A method of providing long term pain management is disclosed herein. In the method, a catheter is surgically implanted to create an infusion site at a peripheral neural structure. An implantable pump and reservoir are surgically implanted in subcutaneous tissue. The pump is then operated to deliver a predetermined dosage of medication through the catheter into the infusion site, whereby pain management is provided.

The catheter is implanted by placing a bore needle in communication with a grounding wire of a nerve stimulator. The bore needle is inserted within a facial sheath of the brachial plexus, and stimulated to verify adequate placement within the facial sheath. An arterial line wire is inserted through the bore needle, grounding the arterial line wire with the surrounding tissue, the arterial line wire then stimulated to verify arterial line location adjacent to the brachial plexus. The catheter is then advanced over the arterial line which is then removed.

A closed system providing long term pain management is also provided. The system comprises a surgically implanted catheter having a discharge portion lying in a neural structure peripheral to the central nervous system. The system also comprises an implantable pump and reservoir located in subcutaneous tissue. A proximal end of the catheter, and the reservoir, are in communication with the pump. The pump is operated to deliver a predetermined dosage of medication through the discharge portion of the catheter into the peripheral neural structure.

### BRIEF DESCRIPTION OF THE DRAWINGS

The foregoing summary, as well as the following detailed description of preferred embodiments, is better understood when read in conjunction with the appended drawings. For the purpose of illustrating the invention, there is shown in the drawings exemplary embodiments of the invention; however, the invention is not limited to the specific methods and instrumentalities disclosed. In the drawings:

FIG. 1A is a diagram illustrating an intercortal or interparietal block in accordance with one embodiment of the present invention;

FIG. 1B is a diagram illustrating a radial or ulnar block in accordance with one embodiment of the present invention;

FIG. 1C is a diagram illustrating a scalene block in accordance with one embodiment of the present invention;

FIG. 1D is a diagram illustrating a tibial block in accordance with one embodiment of the present invention;

FIG. 1E is a diagram illustrating a femoral block in accordance with one embodiment of the present invention;

FIG. 1F is a diagram illustrating an ilioinguinal block in accordance with one embodiment of the present invention;

FIG. 1G is a diagram illustrating a paravertebral block in accordance with one embodiment of the present invention;

FIG. 2 is a flow chart illustrating an exemplary method of peripheral nerve analgesia by using brachial plexus placement through an axillary approach in accordance with one embodiment of the present invention;

FIG. 3 is a flow chart illustrating an exemplary method of peripheral nerve analgesia by way of a thoracic nerve block in accordance with one embodiment of the present invention; and

FIG. 4 is a perspective drawing of an exemplary bore needle in accordance with one embodiment of the present invention.

### DETAILED DESCRIPTION OF THE INVENTION

### Overview

The present invention provides a closed, implantable system that administers analgesic medication to peripheral nerves and plexi. One embodiment of the present invention therefore provides long-term analgesia for localized chronic pain. To implant the system, an anesthetic reservoir/pump is implanted in the subcutaneous tissue of a patient with a connecting catheter that is implanted adjacent to a nerve or plexus, rather than in the spinal canal and central nervous system (CNS), for the purpose of administration of an anesthetic. As may be appreciated, the danger of meningitis, hematoma and systemic effects is decreased as compared to a CNS catheter location.

As may also be appreciated, an embodiment's use of pharmacotherapy enables the treatment of a broader range of pain issues than conventional, electricity-based mononeuropathy treatments. In addition, the delivery of anesthetics to peripheral nerves and plexi enables decreased side-effects as compared to intrathecally-implemented treatments which may require higher doses of anesthetic than is required for peripheral nerves. Also, and according to another embodiment, an implanted system that delivers analgesic medication to peripheral nerves and plexi enables long-term independence and less-frequent patient visits to medical personnel.

One embodiment of the present invention provides that the implanted system be closed, which provides for long term analgesia with decreased risk of infection. As may be appreciated, the present invention provides power analgesia not conventionally available on an outpatient basis.

### Location of Implanted System

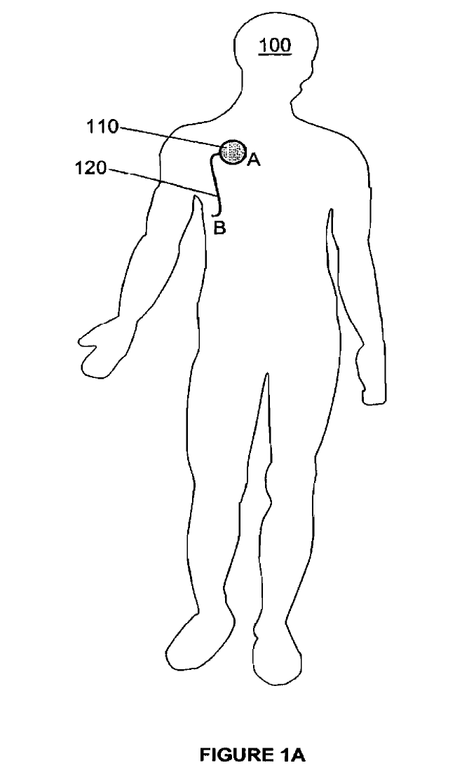

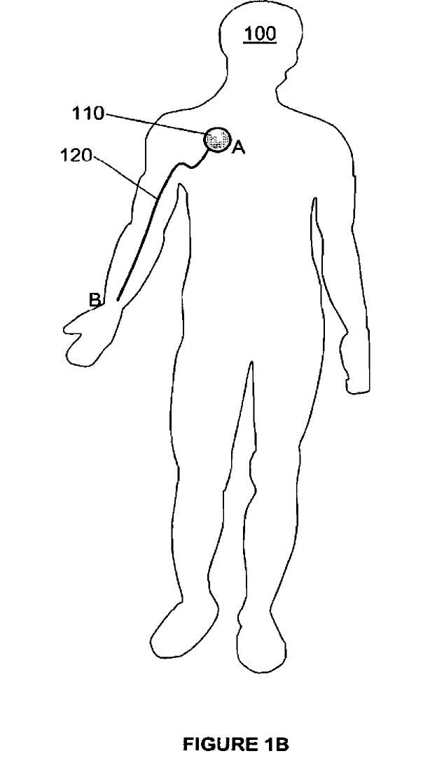

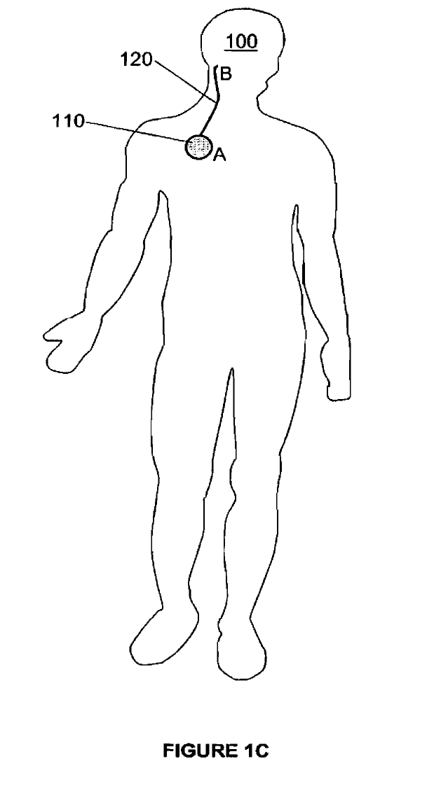

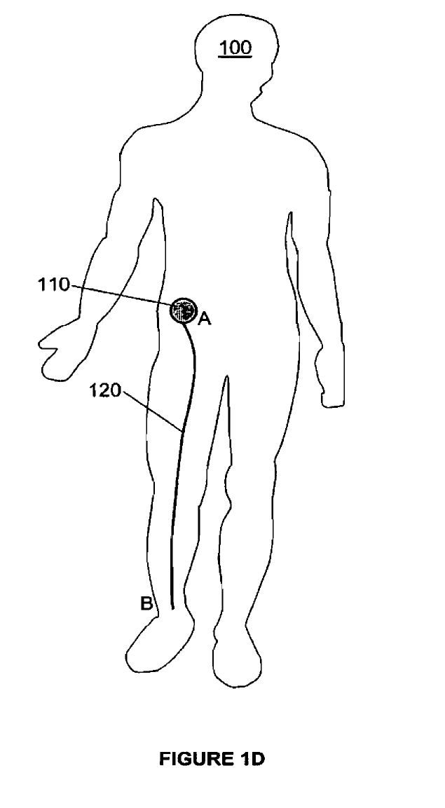

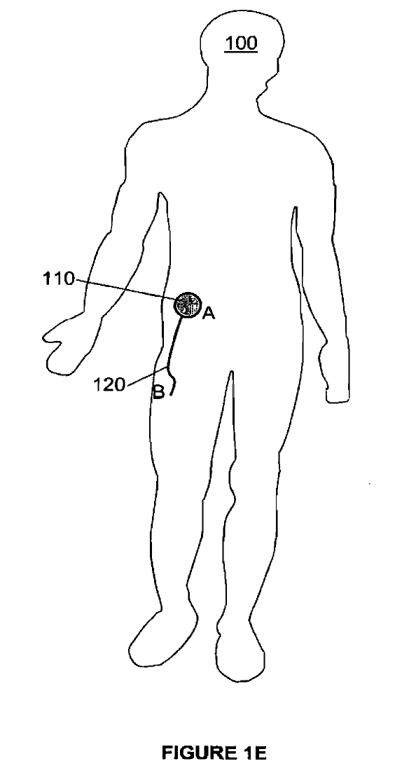

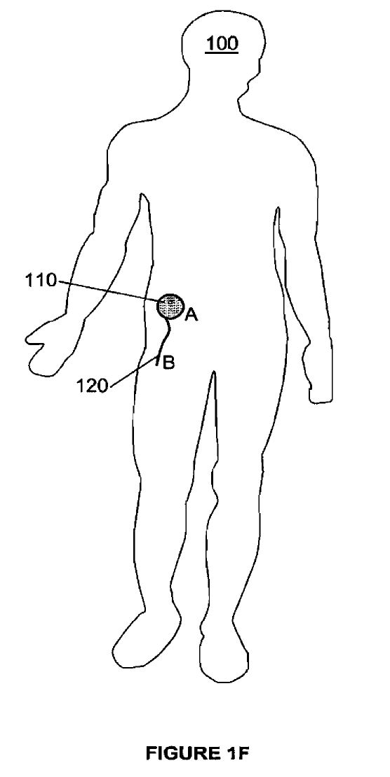

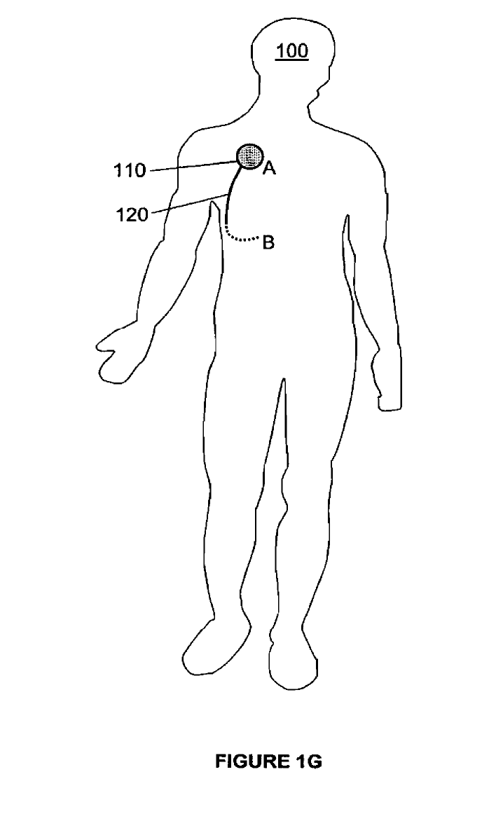

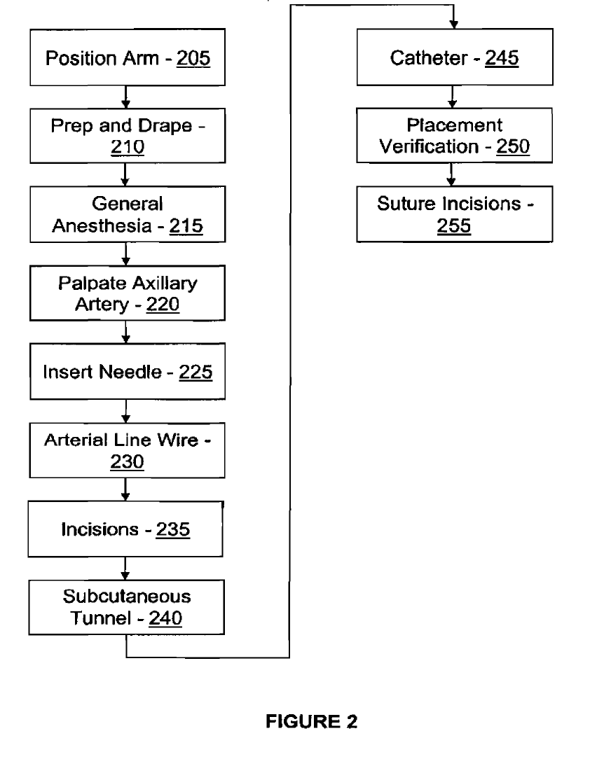

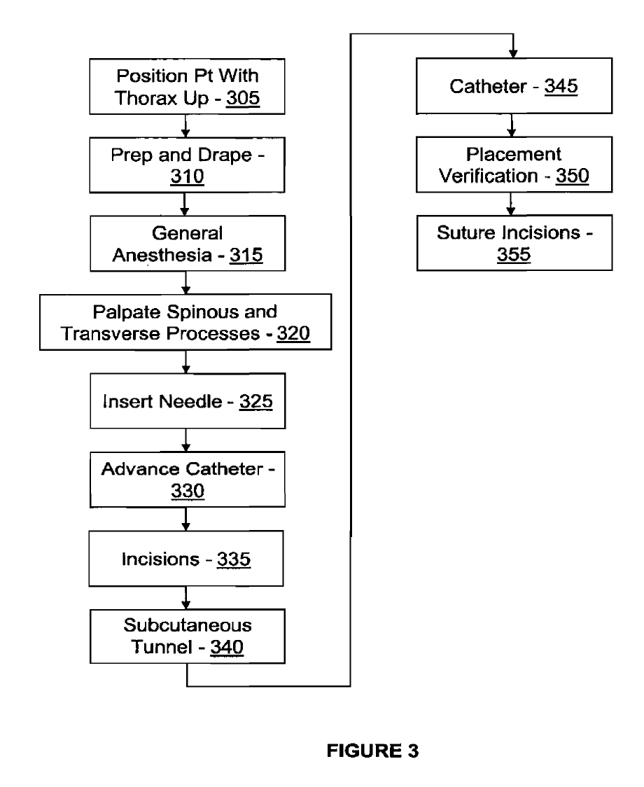

FIGS. 1A-1G illustrate exemplary locations for an implanted, closed anesthetic reservoir/pump and catheter for delivering analgesic medications to peripheral nerves and plexi according to one embodiment of the invention. Turning now to FIG. 1A, a diagram illustrating an intercortal or interparietal block in accordance with one embodiment of the present invention is shown. Within a patient 100 is implanted an anesthetic reservoir/pump 110 at location A. Exemplary methods for such an implantation are discussed below in connection with FIGS. 2 and 3. As may be appreciated, while location A is in the chest cavity of the patient 100, any viable location for the anesthetic reservoir/pump 110 may be used such as, for example, the buttocks or abdomen. In addition, the anesthetic reservoir/pump 110 may be implanted on the patient's 100 left or right side. Characteristics of the anesthetic reservoir/pump 110 will be discussed in greater detail below.

A catheter 120 extends from the anesthetic reservoir/pump 110, through the subcutaneous tissue of the patient 100 and ends adjacent to an intercostal or interparietal nerve—as indicated by location B—to effectuate an intercortal or interparietal block, respectively. According to one embodiment, location A—at which the anesthetic reservoir/pump 110 is implanted—is determined by, for example, the availability of locations within the body that are able to both house the anesthetic reservoir/pump 110 and minimize the amount of subcutaneous tissue through which the catheter 120 must travel.

FIG. 1B is a diagram illustrating a radial or ulnar block in accordance with one embodiment of the present invention. As was the case with FIG. 1A, discussed above, the patient 100 has received the anesthetic reservoir/pump 110 as indicated at location A. The catheter 120 extends from the anesthetic reservoir/pump 110 and terminates at location B, which indicates the radial and/or ulnar area.

FIG. 1C is a diagram illustrating a scalene block in accordance with one embodiment of the present invention. As can be seen, the catheter 120 extends from the anesthetic reservoir/pump 110 at location A to location B, which is located to achieve a scalene block.

FIG. 1D is a diagram illustrating a tibial block in accordance with one embodiment of the present invention. In FIG. 1D, the anesthetic reservoir/pump 110 is implanted at location A, which is now at a lower abdominal area of the patient 100. The catheter 120 extends therefrom to location B, which is placed to effectuate a tibial block.

FIG. 1E is a diagram illustrating a femoral block in accordance with one embodiment of the present invention. Again from location A in a lower abdominal area of the patient 100, the catheter 120 extends from the anesthetic reservoir/pump 110 to location B, which is positioned to effectuate a femoral block.

FIG. 1F is a diagram illustrating an ilioinguinal block in accordance with one embodiment of the present invention. From location A in a lower abdominal area of the patient 100, the catheter 120 extends from the anesthetic reservoir/pump 110 to location B, which is positioned to effectuate a ilioinguinal block.

FIG. 1G is a diagram illustrating a paravertebral block in accordance with one embodiment of the present invention; from location A in a chest area of the patient 100, the catheter 120 extends from the anesthetic reservoir/pump 110 under the subcutaneous tissue on the front of the patient 100 (the solid line portion) around to the back of the patient 100 (the dotted line portion) to location B, which is positioned to effectuate a paravertebral block.

As may be appreciated from FIGS. 1A-G, catheter 120 is placed adjacent to the peripheral nerves or plexi at which an analgesic effect is desired. Although FIGS. 1A-G illustrate several locations for the catheter 120 to deliver analgesic medication, numerous other locations may be used as well, and any such location is equally consistent with the present invention. For example, targets in the head region may include the gasserian gangion, the nasociliary, long ciliary, anterior ethmoidal, subraorbital, supratrochlear, maxillary, infraorbital, spbenopalantine, mandibular, inferior alveolar, lingual, auriculotemporal, masseter and mental nerves. Targets in the neck may include, for example, the cervical plexus, greater and lesser occipital nerves, greater auricular nerve, stellate ganglion and glassopharyngeal nerves. Targets in the upper extremity may include, for example, the brachial plexus: interscalene, supraclavicular, infraclavicular and axillary approach, and the radial, median, ulnar and digital nerves. Targets in the thorax may include, for example, splanchnic nerves, thoracic sympathetic ganglion and intercostals nerves. Targets in the abdomen may include, for example, lumbar sympathetic ganglion, celiac plexus, and ilioinguinal, iliohypogastric and genitofemoral nerves. Targets in the pelvis may include, for example, the sciatic, femoral, lateral femoral cutaneous, obturator, common peroncal, saphanous, tibial, deep peroneal, superficial peroneal and saphaneous and sural nerves.

In one embodiment of the present invention, the catheter 120 is flexible and suitably strong for its intended application. The catheter 120 can be fabricated from a nonreactive material such as, for example, a material such as silicon or polyvinyl plastic. The catheter 120 may be similar to catheters used for intrathecal catheters, or may be a specialized catheter specifically designed for long-term implantation adjacent to a peripheral nerve or plexus. Additionally, an implanted catheter 120 may be lined by a flexible metal strip which would be conducive to electrical conduction. Such a strip could be stimulated to verify catheter placement adjacent to the plexus. As may be appreciated, other embodiments may not have a metal strip.

Regarding the anesthetic reservoir/pump 110, such a pump 110 is, in one embodiment, an implantable fixed or programmable subcutaneous pump or the like. Components designed for epidural or intrathecal delivery may require modification for use in the present invention, depending on the location of infusion and placement of the pump. For example, a reduction in size of the pump 110 may be required in the case of juvenile patients, whereby an adult patient may require a larger pump 110. Also, if one or more anesthetics contained within the pump 110 are particularly potent, the size of the pump 110 may be reduced, while a pump 110 containing more diluted concentrations of anesthetic may need to be increased to contain a sufficient amount of such anesthetic to achieve the desired analgesic effect.

Exemplary pumps 110 that may be used in connection with the present invention, in modified or unmodified form, are the AlgoMed® and SyncroMed® pumps, manufactured by Medtronic, Inc., and Arrow model 3000 pumps, manufactured by Arrow International, Inc. As may be appreciated, such a listing of commercially-available pumps 110 is not all-inclusive as other, general-use or specialized pumps 110 or the like may be implemented in connection with the present invention. Such pumps 110 may be programmable to deliver analgesic medication at a constant rate, boluses, patient controlled boluses and the like. Using Radio Frequency (RF) circuitry, a pump 110 may be programmed from an office or another remote location. In addition, such pumps 110 may contain a reservoir fill port that enables refill of the pump 110 by way of injection.

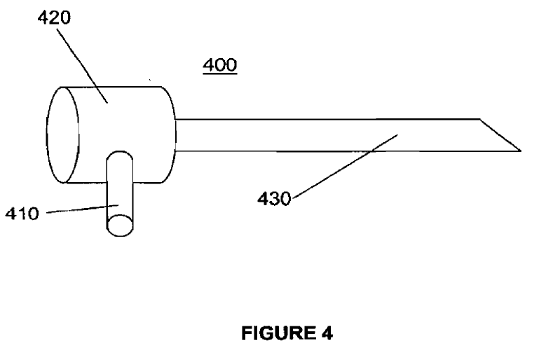

In addition to the catheter 120 and the pump 110, a bore needle, such as, for example, a Crawford needle may be required for implantation of the catheter 120. One exemplary needle that may be used in connection with an embodiment of the present invention is discussed below in connection with FIG. 4. Other embodiments of the present invention can involve other needles for initial injection of a nerve or plexus.

### Method of Peripheral Nerve Analgesia

To illustrate an exemplary implantation according to one embodiment of the present invention, two exemplary methods of peripheral nerve analgesia are disclosed herein: a brachial plexus placement through an axillary approach (discussed below in connection with FIG. 2), and a thoracic nerve block (discussed below in connection with FIG. 3). As may be appreciated, the disclosed methods of peripheral nerve analgesia are in no way all-inclusive, as methods of implanting a system in accordance with the present invention may be used to implant, for example, a pump and catheter in any bodily peripheral nerve or plexus, such as the locations discussed above in connection with FIGS. 1A-G. One of skill in the art would be familiar with general surgery techniques, so detail pertaining to each technique is therefore omitted herein for brevity.

### Brachial Plexus Placement Through Axillary Approach:

Turning now to FIG. 2, a flow chart illustrating an exemplary method of peripheral nerve analgesia by using brachial plexus placement through an axillary approach in accordance with one embodiment of the present invention is shown. At step 205, the patient's elbow is extended and arm abducted to create a substantially 90° angle between the arm and the patient's 110 (not shown in FIG. 2 for clarity) side. At step 210, the axilla and chest wall are prepared and draped in a sterile manner. At step 215, the patient 110 is given general anesthesia with, for example, endotracheal intubation.

At step 220, the axillary artery is palpated in the anterior wall of the axilla and, at step 225, a spinal needle is attached to a bore needle, such as a beveled “block needle” or the like, and the grounding wire of a nerve stimulator. The spinal needle is used, here, to provide electrical conductivity between the bore needle and the nerve stimulator. The bore needle is inserted substantially parallel to or tangentially to the axillary artery until penetrating the facial sheath of the brachial plexus. A stimulator technique is then used such as, for example, a stimulator technique disclosed in Chapter 57 of Steven D. Waldman, ed., Interventional Pain Management, 2nd ed. (W.B. Saunders Co. 2001), which is hereby incorporated by reference in its entirety. The bore needle should be advanced approximately 5 mm to ensure such needle is within the sheath. Local anesthetic is injected with frequent aspiration to ensure the tip is not within a vessel.

At step 230, an arterial line wire is inserted through the bore needle. The arterial line wire is grounded with the surrounding tissue and stimulation applied to verify location adjacent to the plexus. As will be discussed below in connection with FIG. 4, a protrusion may be formed on the needle so as to expedite the stimulation process by obviating the need for a spinal needle to serve as an electrical conductor.

At step 235, incisions are made. For example, an approximately 1 cm incision is made in the skin and subcutaneous tissue at the entry site of the arterial line wire. The arterial line wire is left in place and can be stimulated to verify a location next to the nerve plexus. A horizontal incision is then made in the anterior chest wall. Then dissection such as, for example, blunt/sharp dissection, is utilized to create a subcutaneous pocket. The dissection may take place in a conventional or specialized surgical fashion, as any such method is equally consistent with the present invention. The pump 110 (not shown in FIG. 2 for clarity) is then inserted into the pocket and sutured in place with, for example, nylon sutures or the like.

At step 240, a subcutaneous tunnel is created between the pocket formed above in connection with block 235 and the axillary incision. At step 245, the catheter 120 (not shown in FIG. 2 for clarity) is placed over the arterial line wire using, for example, the Seldinger technique. The wire is then removed. In some embodiments, the wire is impregnated with metal to allow verification of a nerve plexus. The catheter is then thread through the subcutaneous tunnel to the site of the pump 110, and excess catheter 120 length is cut away. The catheter 120 is aspirated to ensure that it is not within a vessel, and then the catheter 120 is attached to the pump 110.

At step 250 verification of the placement of the catheter 120 is made using, for example, stimulation or fluoroscopy. Finally, at step 255, the incision sites are sutured closed.

The aforementioned technique can be utilized with any of the brachial plexus approaches, such as interscalene, subclavian and infraclavicular. The only modification of the above procedure for such approaches may involve the respective initial injection site for accessing the nerve plexus. As may be appreciated, the present invention may be implanted to provide analgesic medication to any bodily peripheral nerves or plexi, such as those listed above in connection with FIGS. 1A-1G. Background information concerning variations and/or modifications to the method illustrated in FIG. 2, as well as to the method of FIG. 3, to be discussed below, to access the different nerves and plexi may be found in Garber J. E., Hassenbusch, S. J. III, Spinal Administration of Nonopiate Analgesics for Pain Management, 2nd ed., (W.B. Saunders Co. 2001); David L. Brown, ed., Regional Anesthesia and Analgesia, 1^{st} ed. (W.B. Saunders Co. 1996); Jordan Katz, ed., Atlas of Regional Anesthesia, 2nd ed. (Appleton and Lange 1994); Chan V. W. S., Continuous Intercostal Nerve Block in Postoperative Pain Management (Churchill Livingston 1993); Chan V. W. S., Ferrante F. M., Continuous Thoracic Paravertebral Block in Postoperative Pain Management (Churchill Livingston 1993); VadeBoncouer T. R., Interpleural Regional Analgesia in Postoperative Pain Management (Churchill Livingston 1993), all of which are hereby incorporated by reference in their entirety. Additional reference to brachial plexus catheter techniques is included in Concepcion M., Continuous Brachial Plexus Catheter Techniques in Postoperative Pain Management (Churchill Livingston. 1993) which is also incorporated by reference in its entirety.

### Thoracic Nerve Blocks

Additionally, intercolstal, interpleural and paravertebral approaches may follow the above procedure with the exception of initial injection and the use of electrical stimulation. As may be appreciated to one of skill in the art, electrical stimulation should not be utilized in paravertebral, intercostal or interpleural blockades.

Turning now to FIG. 3, a flow chart illustrating an exemplary method of peripheral nerve analgesia by way of a thoracic nerve block in accordance with one embodiment of the present invention is shown. At step 305, the patient 100 (not shown in FIG. 3 for clarity) is placed in, for example, the lateral decubitus position with the affected side of the thorax facing up. At step 310, chest and back of the patient 100 are prepared and draped in a sterile manner. At step 315, the patient 100 is given general anesthesia with, for example, endotracheal intubation or the like.

At step 320, the spinous process and transverse process of the desired level of the thorax is palpated. At step 325, a finder needle such as, for example, a 22 gauge finder needle, is inserted substantially perpendicular to the skin and contact is made with the transverse process. Then a spinal needle or the like such as, for example, a 16-18 gauge spinal needle, is inserted in the same location. The spinal needle is walked cephalad off the transverse process' superior border. The needle is angled superiorly and inserted through the superior costotransverse ligament and into the paravertebral space. Aspiration is then performed to check for blood or spinal fluid.

At step 330, a catheter 120 such as, for example, a 20 gauge epidural catheter, is advanced through the bore needle and approximately 2 to 3 em into the paravertebral space. At step 335, an approximately 1 cm incision is made in the skin and subcutaneous tissue at the site of the catheter 120. A substantially horizontal incision is then made in the subcutaneous tissue and skin of the anterior chest wall, abdomen, buttocks or back. Then dissection such as, for example, blunt/sharp dissection is utilized to create a subcutaneous pocket as was discussed above in connection with step 235 of FIG. 2, above. The pump 110 is then inserted into the pocket and sutured in place with, for example, nylon sutures or the like.

At step 340, a subcutaneous tunnel is created between the pocket created in step 335 and the site of the catheter 120. At step 345, the catheter 120 is threaded through the subcutaneous tunnel to the site of the pump 110. Excess catheter 120 length is cut away, and then the catheter 120 is aspirated to ensure that it is not within a vessel. The catheter 120 is then attached to the pump 110.

At step 350, verification of catheter 120 placement takes place using, for example, stimulation or fluoroscopy. Finally, at step 355 the incision sites are sutured closed. As may be appreciated, intercostals and interpleural insertion can be achieved by using the method of FIG. 3. The initial site of insertion should be modified as indicated.

### Surgical Placement of Catheters

Surgical placement of infusion catheters, such as the catheter 120, may follow the basic technique dictated in the placement of implantable peripheral nerve stimulation. This technique is mown in the art of Anesthesia and Neurosurgery, and is discussed in Heavner et al., Peripheral Nerve Stimulation: Current Concepts in Interventional Pain Management, 2nd ed., (W.B. Saunders Co. 2001), which is hereby incorporated by reference in its entirety.

As may be appreciated, the site of afferent pain stimulation should be determined by history and examination. Through surgical technique, the site of mononeuropathy or plexus site should be exposed. The catheter 120 is sutured in place adjacent to the aforementioned nerve or plexus. The catheter 120 may be anchored to, for example, bone, fascia or ligament adjacent to the targeted peripheral nerve or plexi. In such an embodiment of the present invention, an open dissection may be required. Regardless, once the catheter 120 is in place, a track is created in the subcutaneous tissue of the patient 100. A tunnel and connecting pocket is then created as discussed above in connection with the methods of FIGS. 2 and 3.

Referring now to FIG. 4, a perspective drawing of an exemplary bore needle 400 in accordance with one embodiment of the present invention is shown. The needle 400 includes a protrusion 410, a base 420 and a bored metal shaft 430. The protrusion 410 is electrically conductive and extends from the base 420 to create a corner therebetween. The protrusion could, in an alternative embodiment, extend from the metal shaft 430. The protrusion 410 could also be substantially perpendicular to the base 420, and/or the metal shaft 430. Further, the protrusion is conductively continuous with and to the metal shaft 430. The protrusion 410 serves as a contact point for an electrical connector used to stimulate a peripheral nerve or plexus in accordance, for example, with the method discussed above in connection with FIG. 2. Such a connector may be, for example, a “banana” clip or the like. In addition, a metal impregnated catheter 120 (not shown in FIG. 4 for clarity) may be used to conduct an electrical signal to the peripheral nerve or plexus being stimulated. Thus, in one embodiment of the present invention, the needle 400, when connected to an electrical power source by way of the protrusion 410 and in conjunction with a metal impregnated catheter 120, obviates the need for a spinal needle to serve as an electrical conductor. In such an embodiment, therefore, time may be saved in the surgical procedure because of the consolidation of equipment.

### Medication and Dosing

The present invention involves novel drug dosing and utilization. For example, one embodiment requires long term and continuous dosing of analgesics at a peripheral nerve site. Such a chronic dosing schedule is not currently employed in conventional analgesic delivery methods. An embodiment of the present invention may require long-term dosing of analgesics at a peripheral nerve site for days, weeks, months or even years.

Additionally, the analgesics employed by certain embodiments of the present invention may be of diverse types. Bupivacaine is a typical peripheral nerve analgesic, as are similar drugs such as tetracaine and lidocaine. The present invention, however, advocates the use of opioids, antispasmodics, alpha 2 agonists and local anesthetics independently and in combination to promote analgesia.

In one exemplary embodiment, a 42 year old patient with a history of ulnar neuropathy had a catheter surgically implanted adjacent to the symptomatic nerve. A combination of tetracaine, clonidine and baclofen was administered to the peripheral nerve site via an implanted pump system. The patient received doses of 10-25 mg/day of tetracaine, 50-100 mcg/day of clonidine and 50-100 mcg/day of baclofen in combination to achieve analgesia. As may be appreciated, the above disclosed dosing method is in no way all-inclusive, as similar types of drug combinations are possible for peripheral nerves and plexi pain treatment in accordance with the present invention.

Thus, a method and system for using a closed and implanted system for selective infusion of analgesic medicine to peripheral nerves and plexi has been provided. While the present invention has been described in connection with the exemplary embodiments of the various figures, it is to be understood that other similar embodiments may be used or modifications and additions may be made to the described embodiment for performing the same function of the present invention without deviating therefrom. For example, while one skilled in the art will recognize that the present invention as described in the present application refers to specific nerves and plexi, an embodiment of the present invention extends to any peripheral nerve or plexus. Therefore, the present invention should not be limited to any single embodiment, but rather should be construed in breadth and scope in accordance with the appended claims.

What is claimed:

1. A method providing long term pain management, the method comprising the steps of:
surgically implanting a catheter to create an infusion site, wherein a discharge portion of the catheter lies in a peripheral neural structure;
surgically implanting an implantable pump and reservoir in subcutaneous tissue, wherein a proximal end of the catheter, and the reservoir, are in communication with the pump; and
operating the pump to deliver a predetermined dosage of medicament comprising a therapeutic agent selected from the group consisting essentially of opiods, antispasmodics, and alpha 2 agonists, for a period exceeding one week, through the discharge portion of the catheter into the infusion site, whereby the pump and the catheter remain subcutaneously implanted to provided long term, chronic pain management is.

2. The method of claim 1, wherein the neural structure is a brachial plexus nerve complex.

3. The method of claim 2, wherein the catheter is implanted using an axillary approach.

4. The method of claim 2, wherein the catheter is implanted using a subclavian, interscalene or infraclavicular approach.

5. The method of claim 2, wherein implanting the catheter comprises the steps of:
placing a bore needle in communication with a grounding wire of a nerve stimulator;
inserting the bore needle within a facial sheath of the brachial plexus;
stimulating the bore needle to verify adequate placement within the facial sheath;
inserting an arterial line wire through the bore needle;
stimulating the arterial line to verify arterial line location adjacent to the brachial plexus; and
advancing the catheter over the arterial line and removing the arterial line.

6. The method of claim 5, wherein implanting the pump and reservoir further comprises the steps of:
making a first incision in skin and subcutaneous tissue at an arterial line skin penetration location;
making a second incision, creating a subcutaneous pocket, and inserting the pump into the pocket;
creating a subcutaneous tunnel between the pocket and the first incision; and
threading the catheter through the subcutaneous tunnel to the pocket and attaching the catheter to the pump.

7. The method of claim 5, wherein the bore needle has a conductive protrusion located at a base thereof and extending therefrom to create an angle therebetween to facilitate attachment to the grounding wire of the nerve stimulator.

8. The method of claim 1, wherein the neural structure is a gasserian ganglion, a nasociliary nerve, a long ciliary nerve, an anterior ethmoidal nerve, a subraorbital nerve, a supratrochlear nerve, a maxillary nerve, an infraorbital nerve, a sphenopalantine nerve, a mandibular nerve, an inferior alveolar nerve, a lingual nerve, an auriculotemporal nerve, a masseter nerve or a mental nerve.

9. The method of claim 1, wherein the neural structure is a cervical plexus, a greater occipital nerve, a lesser occipital nerve, a greater auricular nerve, a stellate ganglion, a glassopharyngeal nerve, a brachial plexus with the catheter implanted using an interscalene approach, a brachial plexus with the catheter implanted using a supraclavicular approach, a brachial plexus with the catheter implanted using an infraclavicular approach, a brachial plexus with the catheter implanted using an axillary approach, a radial nerve, a median nerve, an ulnar nerve, a digital nerve, a splanchnic nerve, a thoracic sympathetic ganglion, an intercostal nerve, a lumbar sympathetic ganglion, a celiac plexus, an ilioinguinal nerve, an iliohypogastric nerve, a genitofemoral nerve, a sciatic nerve, a femoral nerve, a lateral femoral cutaneous nerve, an obturator nerve, a common peroneal nerve, a saphanous nerve, a tibial nerve, a deep peroneal nerve, a superficial peroneal nerve, a superficial saphaneous nerve or a superficial sural nerve.

10. The method of claim 1, wherein the neural structure is in a thoracic region.

11. The method of claim 1, wherein the neural structure is an intercostal, interpleural, or paravertebral nerve complex.

12. The method of claim 11, wherein implanting the catheter comprises the steps of:
inserting a bore needle into skin and contacting a transverse process;
walking the bore needle cephalad off a superior boarder of the transverse process;
inserting the bore needle through a superior costotransverse ligament and into the paravertebral space; and
advancing the catheter through the bore needle and into the paravertebral space.

13. The method of claim 1, wherein the catheter is implanted using a needle, the needle comprising:
an electrically conductive shaft having a first end adapted to enter a facial sheath of a neural structure, a second end, wherein the shaft has an interior channel running longitudinally therethrough; and
an electrically conductive protrusion extending from the shaft to create a corner therebetween, the protrusion facilitating connection of the needle to a nerve stimulator.

14. The method of claim 13, wherein the protrusion of the needle is adapted to be operatively connected to a clip located at a distal end of a grounding wire of a nerve stimulator.

15. The method of claim 1, wherein the predetermined dosage of medication is approximately between 10-25 mg/day of tetracaine, approximately between 50-100 mcg/day of clonidine, and approximately between 50-100 mcg/day of baclofen.

16. The method of claim 1, where the catheter has an embedded and electrically conductive material throughout the catheter length sufficient to enable electrical conduction, the material facilitating stimulation to verify a catheter distal end location adjacent to the neural structure.

17. The method of claim 1, wherein the medicament further comprises a peripheral nerve analgesic.

18. The method of claim 17, wherein the peripheral nerve analgesic is one selected from the group consisting of bupivacaine, tetracaine and lidocaine.

19. A method providing long term pain management, the method comprising the steps of:
surgically implanting a catheter to create an infusion site, wherein a discharge portion of the catheter lies in a peripheral neural structure;
surgically implanting an implantable pump and reservoir in subcutaneous tissue, wherein a proximal end of the catheter, and the reservoir, are in communication with the pump; and
operating the pump to deliver a predetermined dosage of medicament comprising a therapeutic agent selected from the group consisting of opiods and alpha 2 agonists, for a period exceeding one week, through the discharge portion of the catheter into the infusion site, whereby long term, chronic pain management is provided.

20. The method of claim 19, wherein the medicament further comprises antispasmodics.
